(19) United States
(12) Patent Application Publication
(10) Pub. No.: US 2016/0178526 A1
(43) Pub. Date: Jun. 23, 2016

(54) Optical Sensor Device for Repetitive Assays in Biological Fluids

(71) Applicant: Light Pointe Medical, Inc., St. Louis Park, MN (US)

(72) Inventor: Hiroshi Nomura, Shorewood, MN (US)

(21) Appl. No.: 14/575,781

(22) Filed: Dec. 18, 2014

(51) Int. Cl. G01N 21/77; G01N 33/66

(52) U.S. Cl. CPC G01N 21/7703; G01N 33/66

(57) ABSTRACT

Annular shaped sensor devices are disclosed that comprise an axial array of wedge-shaped optical sensors having tips oriented inward, with analyte-reactive reagents resident on a portion of the tip surfaces. These annular optical sensor devices are cut out from sheets or continuous sheeting of flat optically transparent polymers, and are preferably coated with a cladding material on major surfaces, other than the tips, so as to function as optical light guides. The tips are preferably beveled before deposition of the analyte-reactive reagent. The tips are amenable to deflection movement independently of one another. A method of making the annular optical sensor devices is disclosed and a method of using such devices in the analysis of biological fluid droplets is also disclosed.

### BACKGROUND OF THE INVENTION

1. Field of the Invention

The present invention relates to sensing analyte concentration in a biological fluid, and more particularly to an optical device with multiple sensors for measuring concentrations in biological fluids by optical means, also including a method of manufacture thereof and method of use thereof.

2. Description of Related Art

Optical sensors using waveguides such as optical fibers are very useful in performing analyte assays in biological fluids. U.S. Pat. No. 5,859,937 issued Jan. 12, 1999, to Nomura, for example, describes a minimally invasive medical testing device and method for its use which utilizes a light-conducting optical fiber sensor element having a localized textured site thereon, wherein a reagent is deposited. Interaction of the reagent with an analyte specific to the reagent produces a response, such as development of a colored product, which is detectable by means of a change in characteristics of a light beam transmittable through the optical fiber. By means of the textured site and its increased surface area, the sensitivity of the medical testing device is greatly enhanced. The sensor is particularly useful in blood glucose determinations, requiring smaller blood samples than flat strip devices. Improvements in such optical fiber sensor elements have even further increased their sensitivity. Examples of such improved optical fiber sensor elements may be found, for example, in United States Published Patent Application No. 2009/0219509 published Sep. 3, 2009, in the name of Nomura, United States Published Patent Application No. 2011/0097765 published Apr. 28, 2011, in the name of Nomura and U.S. Pat. No. 8,008,068 issued Aug. 30, 2011, to Nomura.

Handling of individual optic fibers during manufacture is not without difficulty in a practical industrial process. Steps would normally include cutting optic fibers into short lengths, attaching them to some form of belt or carrier, exposing a belted bundle or continuous array of optic fiber ends to an etchant such as a stream of atomic oxygen gas, then depositing a mixture of analyte-reactive reagent and hollow polymeric particles on the treated tips, followed by drying and repositioning of such treated fibers into cartridges for use by a consumer such as a diabetic patient. Improvements in optical sensor elements to enhance their manufacturability and improvements in the methods of manufacture thereof are desirable. Such an improvement was disclosed by the Nomura in a United States patent application titled Optical Sensor Element for Analyte Assay in a Biological Fluid, and Method of Manufacture Thereof, wherein a method was described of forming a great many shaped optical wafers from a sheet of optically transparent material, each wafer being in the form of a thin and essentially flat piece of optical material having a narrow cross-sectional width relative to length, and a sharply narrowed tip at one end, approximating the same sensing tip area as an optical fiber tip may ordinarily possess. Included in that improvement was the process of generating a great many optical sensors as individual wafers and loading the individual wafers into suitable cartridges for mounting in user-operated devices, combining this with a system for educing a fluid sample to be expressed from a living organism and contacting the narrowed tip of the wafer with the fluid sample for assay of a fluid component, such as glucose in blood. A significant advance beyond the concept of this previous application is now disclosed in the current specification and claims that follow thereafter.

### BRIEF SUMMARY OF THE INVENTION

One embodiment of the present invention is a optical sensor device for multiple determinations of an analyte or analytes in a biological fluid, comprising a plurality of flat optical waveguides axially arranged to form an annulus having an inner periphery and an outer periphery, each flat optical waveguide being generally in the shape of a wedge having two parallel major surfaces as of a planar sheet or sheet-like source and further having an edge contiguous to the major surfaces, a portion of the edge being a tip oriented toward a central focal point of the annulus and forming a portion of the inner periphery, a portion of the edge oriented outward and forming a portion of the outer periphery of the annulus, further portions of the edge being two lateral sides of the wedge radially oriented outward from the central focal point of the annulus, the wedges being partially separated one from another along the lateral sides, wherein an analyte-reactive reagent is resident as a deposit on the tips, wherein also the major surfaces are coated with cladding and each of the lateral sides are at least partially coated with cladding, the cladding contributing to efficiency of the planar wedges as optical waveguides.

Another embodiment of the present invention is an optical sensor device comprising a plurality of flat optical waveguides axially arranged to form an annulus having an inner periphery and an outer periphery, each flat optical waveguide being generally in the shape of a planar wedge connected on either side to other wedges, yet partially separated from those neighboring wedges by cuts made laterally through a portion of the annulus width, further having a portion of the edge being a tip oriented toward a central focal point of the annulus and forming a portion of the inner periphery, a portion of the edge opposite to the tip being oriented outward and forming a portion of the outer periphery of the annulus, wherein an analyte-reactive reagent is disposed on the tips, wherein also the major surfaces are coated with cladding and each of the lateral sides is at least partially coated with cladding, wherein the tips are textured, the tips being movable independently of one another by deflection as by means of an exterior force.

Another embodiment of the present invention is an optical sensor device comprising a plurality of flat optical waveguides axially arranged to form an annulus having an inner periphery and an outer periphery, each flat optical waveguide being generally in the shape of a planar wedge connected on either side to other wedges, yet partially separated from those neighboring wedges by cuts made laterally through at least a portion of the annulus width, further having a portion of the edge being a tip oriented toward a central focal point of the annulus and forming a portion of the inner periphery, a portion of the edge opposite to the tip being oriented outward and forming a portion of the outer periphery of the annulus, wherein the tips are beveled so that the beveled surface is at a slant to the plane of the wedge and an analyte-reactive reagent is disposed on at least a portion of the slanted area of the tips, wherein also the major surfaces are coated with cladding and edge portions formed by cuts made laterally through a portion of the annulus width are at least partially cladded, the tips being movable independently of one another by deflection as by means of an exterior force.

A significant feature of these various embodiments is the presence of multiple test areas operable independently of one another, yet connected into an optical sensor device of annular shape, allowing the user thereof to mount a single such device into a holder device such as also contains a lancing device, and to run fluid analyses repetitively, such as once per day, twice per day, thrice per day, or on any of several time intervals in a day or in a multiple day period, without going through the inconvenience of reloading a sensor strip into a holder device after each individual test.

### BRIEF DESCRIPTION OF THE DRAWINGS

FIG. 1 is a drawing of an optical sensor device having multiple sensing tips.

FIG. 2 is a drawing of an optical sheet showing outlines of prospective optical sensor devices to be excised from a sheet of optically transparent material.

FIG. 3 is a drawing of an optical sensor device having seven beveled sensor tips.

FIG. 4 is a flowchart of a method of manufacture of optical sensor devices.

FIG. 5 is a drawing illustrating deflection movement of a tip by means of an external shaft.

### DETAILED DESCRIPTION OF THE INVENTION

Various optical fiber sensor elements are described in U.S. Pat. No. 5,859,937 issued Jan. 12, 1999, to Nomura, United States Published Patent Application No. 2009/0219509 published Sep. 3, 2009, in the name of Nomura, United States Published Patent Application No. 2011/0097755 published Apr. 28, 2011, in the name of Nomura, and U.S. Pat. No. 8,008,068 issued Aug. 30, 2011, to Nomura, all of which are incorporated herein in their entirety by reference thereto, and collectively referred to herein as the Nomura patent documents. The optical sensor devices described herein possess many of the analytical advantages of these earlier optical fiber sensor elements, including a one-step testing process for analyte measurement, analyses of analytes in very small sample volumes in contact with a very small tip (a blood sample size of only around 0.1 microliter in diabetes blood glucose testing, for example), rapid test results of potentially two seconds or less, and elimination of hemoglobin interference in tests on blood. However, in addition, such optical sensor devices as described herein are also suitable for manufacture by highly efficient and cost-effective methods which avoid some of the manufacturing and handling disadvantages that arise in the manipulation of short lengths of optical fibers, thereby substantially reducing manufacturing costs.

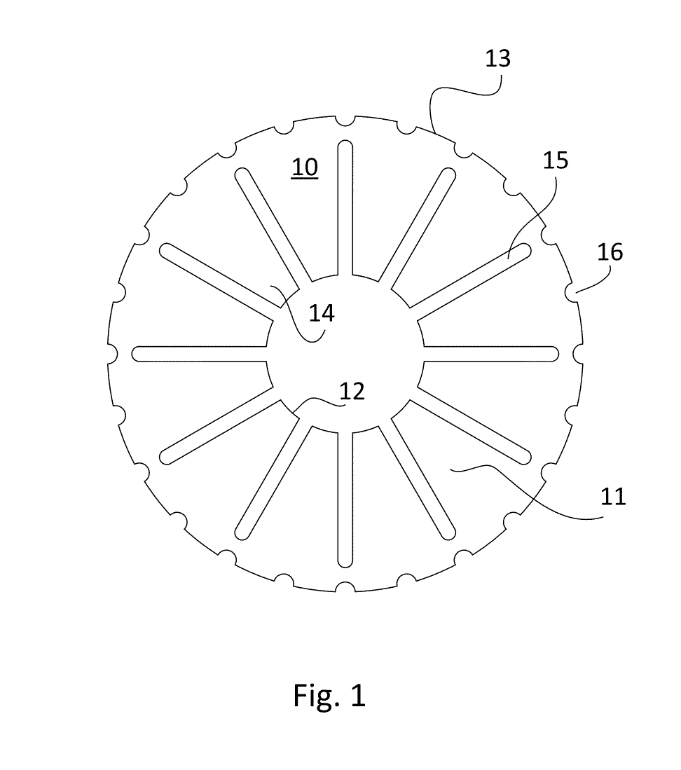

One embodiment of the present invention is illustrated in FIG. 1. An optical sensor device 10 for multiple determination of an analyte in a biological fluid, comprises a plurality of flat optical waveguides 11 in an axial arrangement forming an annulus having an inner periphery 12 and an outer periphery 13, each flat optical waveguide 11 being generally in the shape of a wedge having two parallel major surfaces corresponding to the surfaces a sheet of material from which the sensor element is fashioned. Each wedge comprises a narrowed proximal end 14 defined herein as a tip, wherein the plural array of the tips collectively form a portion of the inner periphery 12 and a wide distal end wherein the distal ends of the wedges collectively form a portion of the outer periphery 13 of the annulus. Cutouts 15 are preferably located extending radially through a portion of the width of the annulus and thereby achieving partial separation of the wedges, and are particularly advantageous for separating the tips 14 one from another. The major surfaces and the edges contiguous with the two surfaces, with the exception of the tips, are preferably coated with a cladding so as to enhance the waveguide characteristics of the sensor element. Edges on the tips, however, are preferably free of any cladding and are textured and coated with a deposit of an analyte-reactive reagent similarly as employed in optical fiber sensors previously disclosed by Nomura. Optionally, indentations or notches 16 are located in the outer periphery to act as registration sites for mechanical engagement and movement of the optical sensor device, such as in rotating the sensor element when mounted within a blood testing device such as a blood glucose monitor, wherein the tips may be advanced from one testing point to another in circular movement. Also, while capability exists in the annular optical sensor device to perform multiple determinations of an analyte in biological fluids, any specific number of determinations may be made per device, whether only a single determination of an analyte or multiple determinations up to the limit of the number of sensor tips resident in the optical sensor device, depending on a user's prerogative and motive. Repetitive analyses or assays of analytes in biological fluids may thus be achieved with sensors of this type, resulting in savings and convenience to the user.

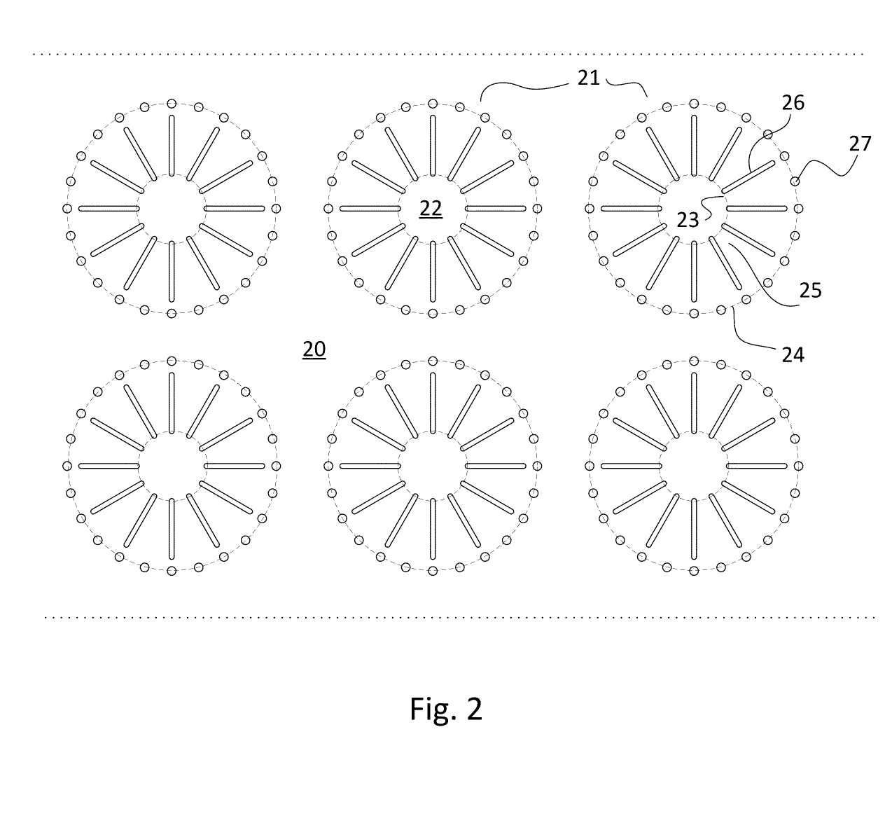

FIG. 2 shows an arrangement whereby annular optical sensor devices may be marked and excised from a sheet of a film of optically transparent material. A sheet 20 has a plurality of annular optical sensor devices 21 designated thereon, wherein radially extending (i.e. radial) cutouts 26 are marked, the location of apertures 27 are marked to be cut out to develop subsequent notches in the outer periphery, and central cores 22 are outlined for removal to expose the inner peripheries 23 composed principally of tips 25 of the wedges, the inner peripheries being illustrated in FIG. 2 by a dashed circle. Also shown is the location of circumferential cutting lines 24 corresponding to the outer peripheries of the devices, again illustrated by dashed circles. The devices depicted in FIGS. 1 and 2 disclose twelve sensor wedges in axial arrangement, but the number of devices may range widely from as little as three to as many as fifty, being limited by preferred diameters of the devices and the number of operable tips that can be contained as independently operable sampling/testing sites or probes within practical constraints of the preferred device diameters.

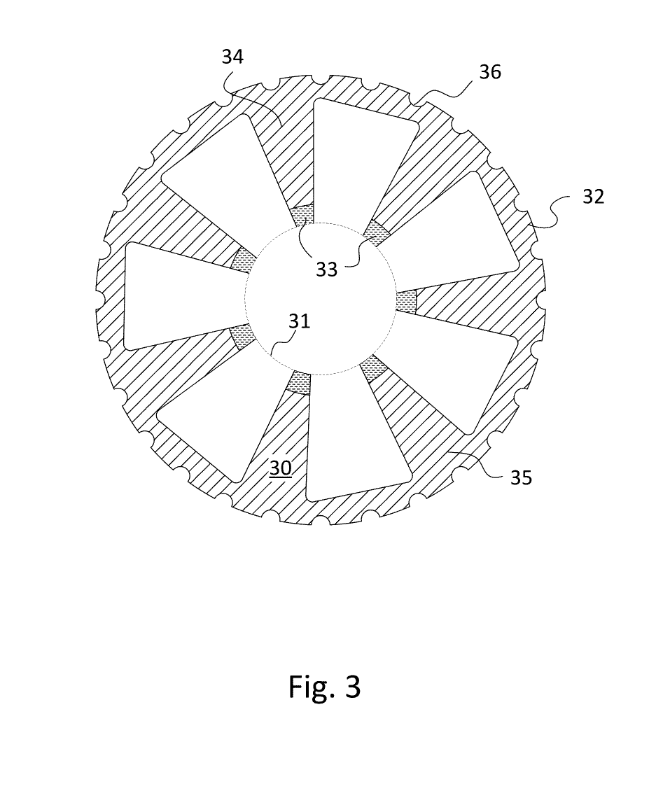

In one alternate arrangement the number of wedges or generally wedge-shaped sensors may be seven, which is advantageous for use in a monitoring program where one fluid analyte test per day is to be performed, the annular sensor device thus providing one week's worth of monitoring an analyte. An optical sensor device having seven sensor tips is illustrated in FIG. 3. Shown in FIG. 3 is an optical sensor device that has been presumably and advantageously cut from a sheet of planar optically transparent material, the device 30 having an inner periphery indicated by the dashed circular line 31 and having an outer periphery 32, and having a set of tips whose inner edges correspond to and constitute a portion of the inner periphery 31. The tips are located at the inner or proximal ends of planar wedges 34 that are shaped by cutting out a portion of the planar material of the annulus. The tips are preferably beveled so as to have a portion 33 of the surface at a slant relative to the otherwise flat surfaces of the planar wedges. Bridging material 35 preferably remains between the wedges to hold them in fixed position within the optical sensor device. Notches 36 are optionally located along the outer periphery, i.e. distal ends of the wedges, and provide a means for locating and moving the device circularly in a holder device such as also contains a lancing device. Other means rather than the notches 36 may be employed to register and rotate or otherwise position the annular device during use. Space is advantageously provided between the lateral edges of the wedges for travel of lancets orthogonally to the plane of the device without striking the device surfaces. While an optical sensor device with seven tips appears well suited for a week's use in once daily testing, other multiples may be advantageous where testing is done on a scale other than once daily. For example, in another alternate arrangement may employ a number of wedges constituting a multiple of two for use in a monitoring program where two fluid analyte tests per day are to be performed. The term wedge is used according to a generally normal definition describing a piece of material thick at one edge and tapering to a thin width at an opposite edge. In this disclosure, it is to be understood that the width of the planar wedges may be varied, as one may infer from differences in cutout widths when comparing FIG. 1 with FIG. 3. The shape of the planar wedges may vary thusly from wide to narrow, though within a specific optical sensor device it is generally preferable that all wedges be of the same dimensional shape.

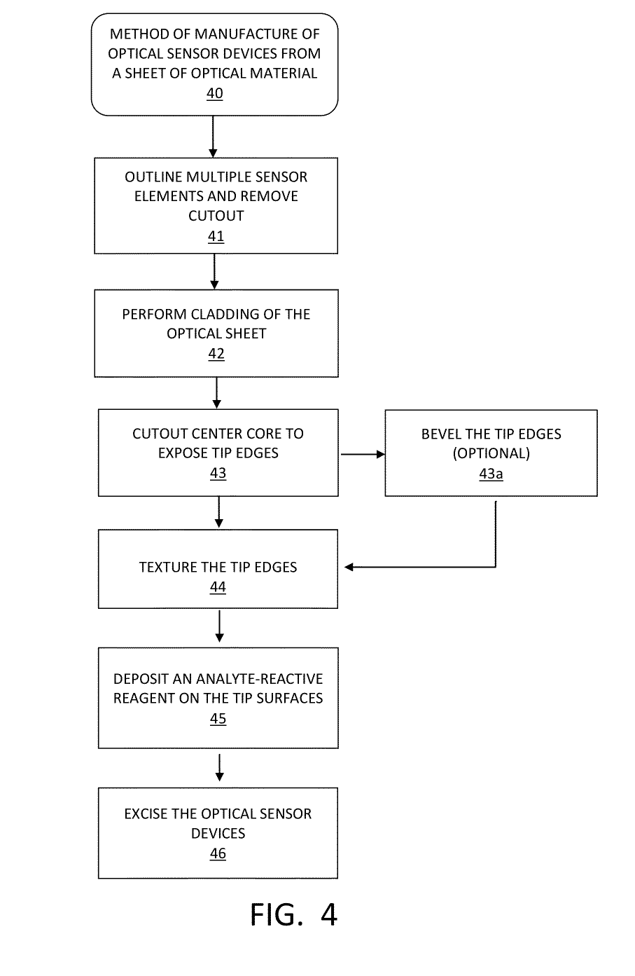

FIG. 4 shows a roadmap illustrating a series of steps that may be taken to generate optical sensor devices of the invention. In a first step 41, a sheet or continuous length of sheeting of optically transparent material is modified such as by being passed through a rotary die cutting operation whereby a multiplicity of sensor elements are outlined as by a series of sets of cutouts that radiate from focal points through a portion of what will subsequently become the widths of the annuli. These correspond to cutouts 15 shown in FIG. 1. Conveniently, but optionally, a series of apertures, correspondingly leading to the notches indicated by 16 in FIG. 1, may, in the same step, be excised from what will subsequently become the outer periphery of each sensor element, these being advantageously coordinated spatially with each radial array of cutouts 15 (cf. FIG. 1). In a following step 42, the sheet or continuous sheeting is coated with a cladding material, covering the two major surfaces of the planar wedges as well as exposed edges contiguous with the two major surfaces as generated by cutouts and excised apertures. After the cladding material is applied, the next step 43 preferably involves excising the center cores of the intended annuli, thereby exposing tip edges that now compose a portion of the inner periphery. Thereafter, texturing of the exposed edge of the tips such as by etching processes may be accomplished as in step 44 to provide a textured surface zone. However, an optional step 43a may intervene before step 44 in which each tip edge is beveled so as to be slanted relative to the plane of the two major surfaces that correspond to the top and bottom surfaces of the starting sheet material. The texturing step is advantageous in that it provides a surface to accommodate a larger amount of analyte-reactive reagent than would be normal for a smooth surface. However, texturing is not a strictly necessary feature of the present invention, and this texturing step is in some applications an optional step. Upon completion of the texturing step 44, when practiced, it is advantageous to apply a deposit of an analyte-reactive reagent to the exposed, textured surfaces of the tips as in step 45. A following step 46 would involve excising the annular optical sensor devices from the sheet or sheeting by means of circular cuts corresponding to the outer peripheries of the sensor elements, completing the formation of the sensor annuli. The sequence of steps 45 and 46 may be reversed, though the change in order of these steps depends upon methods and means employed in depositing the analyte-reactive reagent on the tip surfaces.

Light guides use cladding on their optical material to confine the light and to minimize loss of light through the outer walls. Although cladding of the optical sheeting may be employed before or after the cutting steps, it is advantageous to perform the cladding operation after certain cutouts are removed, including the apertures 16 but most particularly the cutouts 15 (see FIG. 1). It is preferable that the edges (i.e. sidewalls) of the prospective wedges exposed by removal of the cutouts 15 be cladded so as to minimize sideways escape of lighting and improve optical performance of the resulting wedge waveguides during application in analyte testing. Cladding may be performed by dip-coating, spraying, roller-coating, or similar such methods. A particularly suitable cladding technique is deposit of a cladding material by plasma polymerization. Plasma polymerization provides for pinhole-free coatings and also achieves penetration into embossed or perforated recesses. Effective cladding compounds will usually have an index of refraction that is significantly different than the refractive index of the optical material being coated. Fluorocarbon-containing coatings are particularly preferred. Fluorocarbon coatings may be formed through plasma polymerization of fluorine-containing monomers such as tetrafluoroethylene, hexafluorobenzene, or hexafluoroethylene. Combinations of these monomers with each other and with other chemical monomers, both fluorocarbon and nonfluorocarbon, may be employed in a plasma coating process, and cladding with a coating containing little or no fluorinated species may be also employed, though this is not generally as effective as the aforementioned fluorine-containing monomers as cladding compounds. Chemical cladding is not necessarily required. In some applications, so-called “air-cladding” may suffice, in that the index of refraction of air differs sufficiently from that of a polymeric material serving as optical wave guide. However, for applications involving insertion of the annular optical sensor devices into mechanical apparatus for biological fluid testing, cladding such as with a fluorocarbon composition is generally preferred versus relying on air cladding.

The optical sensor devices themselves may be formed from any of many suitable optically transparent compositions. Suitable choices include polymeric compounds such as celluloids, cellulose acetates, polyesters, polystyrenes, polymethacrylates, polyolefins, halogen-containing polyolefins, polysulfones, polycarbonates, polyacrylonitriles, and copolymers or terpolymers of these different compositions. Particularly suitable choices include polycarbonates, methyl methacrylate polymers and copolymers, polystyrenes, and styrene-acrylonitrile copolymers. Continuous sheeting of most of these various compositions is available and amenable to being processed through die-cut roller machines. These various choices are governed by the requirement that the resulting annular optical sensor devices have optically transparent bodies that can funnel a light beam from a source adjacent the outer edge (distal edge) on toward a tip (proximal edge), then return a beam of reflectance light to a sensing element suitably located adjacent the outer (distal) edge, the light having traveled to an analytical zone located on the surface of at least one tip at the inner periphery. In cases where continuous sheeting is unavailable but discreet sheets are available for certain polymeric compositions, sheets of such polymers may be handled and processed by means of various types of stamping and die cutting equipment. Processing may be done at room temperature or at elevated temperatures. For sheeting where rigidity of the polymer composition may present processing difficulty, elevated temperatures sufficient to soften the polymer composition may be employed.

As previously shown in FIG. 4, it is advantageous after the initial die cutting and cladding operations to remove the center cores of the planned annuli. This can be accomplished by any of several methods, including die cutting, drilling, shearing as with punching apparatus, or cutting with blades or by laser beams. One or more of these methods may be used in combination. The general purpose of this process step is to generate exposed tips that may be subsequently textured and modified with one or more reagent compositions. Beveling of the tips may be performed in conjunction with this step of the operation. Various means are available to create a beveled surface at the tips of an optical sensor device contiguous to the inner periphery of the device. Material may be removed by sanding or abrasion, by laser sculpting, by contact with slanted cutting edges such as a rotating drill bit, or by shearing with a circular die cutting tool at the tips where the tips are supported by a shaped platen. In construction, welding, and various other industries, a bevel angle is the angle created by the prepared edge of a member and a plane perpendicular to the surface of the member. The same definition is applicable in the following discussion. The angle of the bevel relative to the plane of the wedge may be anywhere from 10 to 80 degrees. A preferred range of the bevel angle would be 50 to 70 degrees. Most preferred is a bevel angle of about 60 degrees. Such an angle results in a tip surface area that is calculated to be about twice the area of the tip edge if no beveling were introduced. In use of the optical sensor device in a fluid testing device, the beveled surface would preferably face the site of expression of a fluid droplet to be analyzed.

The sheet or sheeting with center cores removed may be passed through a texturing operation wherein the tips are subjected to a texturing treatment to provide a textured surface zone having a greatly increased surface area for deposit of an analyte-reactive reagent. This texturing treatment is optional and may be advantageously employed when a stronger signal is desired to be generated in a chromatic analysis method for increased accuracy in an assay. Texturing may be performed using various techniques. One particularly effective technique involves texturing by means of a directed beam of atomic oxygen, as set forth in U.S. Pat. No. 5,560,781 issued Oct. 1, 1996 to Banks et al., which is incorporated herein in its entirety by reference thereto. Atomic oxygen may be used to microscopically alter the surface morphology of polymeric or plastic materials in space or in ground laboratory facilities. For polymeric or plastic materials whose sole oxidation products are volatile species, directed atomic oxygen reactions produce surfaces of microscopic cones. However, isotropic atomic oxygen exposure results in polymer surfaces covered with lower aspect ratio sharp-edged craters. Isotropic atomic oxygen plasma exposure of polymers typically causes a significant decrease in water contact angle as well as altered coefficient of static friction. Atomic oxygen texturing of polymers is further disclosed and the results of atomic oxygen plasma exposure of thirty-three (33) different polymers, including typical morphology changes, effects on water contact angle, and coefficient of static friction, are presented in an article by Banks et al., “Atomic Oxygen Textured Polymers”, NASA Technical Memorandum 106769, Prepared for the 1995 Spring Meeting of the Materials Research Society, San Francisco, Calif., Apr. 17-21, 1995, which hereby is incorporated herein in its entirety by reference thereto. Surfaces with somewhat similar textures may be achieved by exposure to radiofrequency gas plasmas employing oxygen or air as the plasma generating gas.

In this regard, organopolymeric plastics amenable to oxidation and etching by atomic oxygen are preferred for use in the preparation of sensors based on the annular optical sensor devices described herein, such plastics also needing to display good transparency characteristics toward light frequencies intended to be used in subsequent sensor devices. Some examples of such plastics include poly(methyl methacrylate), polystyrene, styrene-acrylonitrile copolymer, various methyl methacrylate copolymers, and polycarbonate. It is most preferable that surfaces to be textured are not coated with fluorocarbon cladding, as such coatings would generally interfere with the texturing development. At the same time, cladding of major surfaces up to the demarcation line of the tip edges to be textured is desirable from the aspect of avoiding texturing beyond the desired surface area. Stray texturing can potentially lead to some leakage and loss of light from an optical beam being passed through the optical waveguide.

The sheets or sheeting having exposed and optionally textured tips are further processed in an analyte-reactive reagent deposition operation, which may include the deposition of hollow microspheres for enhanced sensitivity, in accordance with the aforementioned Nomura patent documents. An analyte-reactive reagent for the analyte is disposed on the tips of the annular optical sensor devices, and a plurality of light scattering particulate bodies may be dispersed through the analyte-reactive reagent, the particulate bodies being adapted to contribute to reflectance of light from the optical material body of the sensor elements back into the optical material body of the sensor elements through at least a portion of the analyte-reactive reagent, when the analyte-reactive reagent is in reaction with the analyte. In particular, use is made of hollow polymeric microspheres, which provide for significantly augmented reflectance of a light beam emitted from the optical material into the sampling zone. Further described is a device wherein exists a sampling zone associated with an analyte-reactive reagent wherein a portion of the reagent is disposed between the surface of the optical material and the hollow polymeric microspheres, the hollow polymeric microspheres providing for reflectance of a light beam emitted from the optical material into the sampling zone. Polymeric hollow microspheres that have proven particularly useful and effective for this purpose are made according to procedures disclosed in U.S. Pat. No. 6,020,435 issued to Blankenship et al. Feb. 1, 2000, which is incorporated herein in its entirety by reference thereto. Deposition of the analyte-reactive reagent containing also the hollow polymeric microspheres is typically followed by a drying operation, thus providing a shelf-stable form of reagent resident on the tips of the annular optical sensor device.

In some applications, texturing as a process step may be bypassed. A useful function of the textured surface is to provide more capacity for a deposit of analyte-reactive reagent so as to improve sensitivity of the device to analyte concentration. However, the use of the hollow polymeric microspheres greatly increases the amount of reflectance light being redirected through the wedge, thus increasing analytical sensitivity and alleviating the need for high loading capacity of the analyte-reactive reagent on the sensor tip surface.

A finishing step in a process of preparation of the annular optical sensor devices is the cutting out of the devices from the sheet or sheeting in which they are contained. This may be accomplished by die cutting, punching, shearing, or any customary method of excising the devices. Methods are preferred, however, whereby the outer periphery edges are capable of receiving a beam of light from a source, and returning a reflectance light beam back outward to a light analysis device without undue loss of light signal strength.

These optical sensor devices may have thicknesses in the range of 1 micrometer (μm) to 10,000 μm, preferably in the range of 10 μm to 1000 μm, and more preferably in the range of 200 μm to 700 μm. Polymeric films or sheeting having thicknesses in the range of 276 μm to 552 μm are particularly preferred. The diameter of the annular optical sensor devices may vary in the range of 10 to 1000 millimeters (mm), more preferably in the range of 15 mm to 100 mm, most preferably in the range of 18 mm to 26 mm. By diameter is meant twice the distance from the central focal point of the annulus to the outer periphery of the annulus. The preferred range in diameter is generally limited on the short end of the range by the need for enough tip space for adequate analysis and light reflectance, and limited on the long end of the range by the constraints of suitable mechanical devices for diagnostic testing. In the case of blood glucose testing, for instance, a handheld medical testing apparatus accommodating an optical sensor device would generally operate most advantageously with devices of 15 mm to 20 mm in diameter. The number of planar wedges contained within an annular optical sensor device may vary from three to fifty, depending upon the diameter of a mechanical device for which the device is designed to be used. Preferably, from five to fifteen wedge-shaped sensors would be contained in the annular optical sensor device. In FIG. 1, the optical sensor device contained twelve wedge-shaped sensors, but this is for purpose of illustration rather than being a limitation. The depth of the sensor, that is, the distance from tip to outer edge, is a function of the diameter range and the size of the central core cutout. The diameter of the central core cutout is preferably around 5 mm to 8 mm, the smaller dimension being appropriate for smaller devices and the larger dimension being appropriate for larger devices.

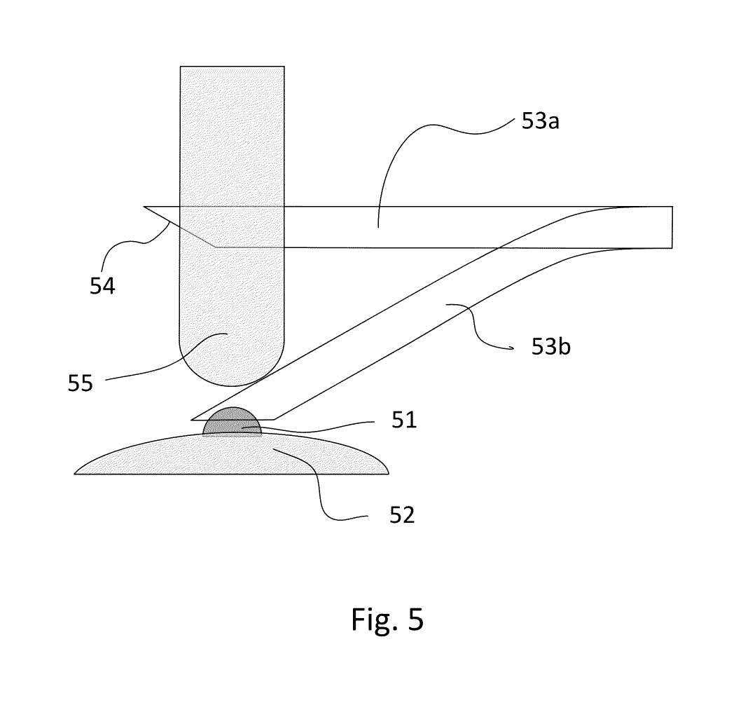

In a practice of using the optical sensor device in a manner envisioned as most preferred at this time, the device would be mounted or otherwise coordinated with a medical testing apparatus, the apparatus being preferably equipped with a lancet as well. The lancet would be actuated in order to obtain expression of a fluid droplet from a puncture wound in the skin of a living subject. A wedge tip having a beveled surface portion, optionally textured, further having an analyte-reactive reagent deposited and resident on the beveled surface, would be positioned so as to hover over the expressed fluid droplet. A movable shaft would be actuated so as to force the wedge tip downward into contact with the droplet of fluid to be analyzed, the wedge being amenable to bending in this step. A light beam of a suitable frequency or range of frequencies is transmitted through the outer periphery edge of this particular wedge opposite from the tip, being transmitted to the tip and into the sampling zone comprising the analyte-reactive reagent deposit in the beveled area on the tip. Changes in the spectral nature of the light beam advantageously occur as a function of optically detectable changes in the reagent sampling zone due to interaction of the analyte-reactive reagent with an analyte in the fluid to be analyzed. A portion of the beam of light is returned as a reflectance from the sampling zone back to the outer periphery edge, where the reflectance light may be sensed and analyzed for spectral changes correlative with concentration of the analyte in the fluid sample. FIG. 5 illustrates one advantageous feature of optical sensor tips that are beveled and the beveled area coated with an analyte-reactive reagent. Shown in FIG. 5 is a small fluid droplet 51 expressed from the skin 52 of a living subject by prior contact with a lancet. A tip of a sensor 53a having a beveled surface 54 contiguous to the inner periphery of the sensor device is located above the site of the fluid droplet and is deflected 53b into contact with the fluid droplet by means of a shaft 55 pressing against the tip or against a portion of the planar wedge at a point set back from the tip. A light beam traveling through the length of the body of the sensor to the beveled tip surface would encounter an analyte reaction zone, and a portion of the light beam thus chromatically altered would be redirected back through the body of the sensor to a light sensing device located external to the sensor. In the most preferred manner envisioned in this disclosure, the beveled tip would be a part of an annular sensor device mounted in a testing device, wherein the beveled tip could be rapidly and accurately positioned over a lancing site for analysis of a fluid droplet amounting to as little as a miniscule 0.1 microliter.

When detecting the spectral changes using reflectance spectroscopy, any part of this light beam that radiates into and through the sampling zone does not generally reenter the optical wedge and does not contribute to the measurement. The technique applied here and described in detail in the aforementioned United States Published Patent Application No. 2009/0219509 published Sep. 3, 2009, in the name of Nomura, United States Published Patent Application No. 2011/0097765 published Apr. 28, 2011, in the name of Nomura and U.S. Pat. No. 8,008,068 issued Aug. 30, 2011, to Nomura, enhances the amount of the light beam that reenters the wedge, being then returned to the outer periphery edge for subsequent analysis. This technique for enhanced reflectance is optimally in the form of particles that scatter light. These particles may be composed of inorganic or organic materials. Inorganic particles useful as reflectance useful as reflectance enhancing agents include silicates and related glasses, and may be in the shape of beads or similarly spherical shapes. Organic particles useful as reflectance enhancing agents include natural and synthetic polymers of various compositions, and may also be solid or hollow.

Hollow beads are particularly effective. The aforementioned U.S. Pat. No. 6,020,435 discloses a process for making organic hollow beads of a nature and polymeric composition that are well suited to this application. So that the analyte-reactive reagent may be accessed by the fluid sample, these reflectance enhancing particles are desirably not film-forming, meaning that a coating or array of these particles does not form a film impenetrable to fluid transport. These particles are associated with an analyte-reactive reagent coating preferably by co-deposition as a mixed coating on the surface of an optical sensor material so as to be present in the reagent sampling zone. In practice, the analyte-reactive reagent is present as a coating intimately in contact with a surface of the optical material and the suitable reflectance enhancing particles are in contact with the reagent layer but optimally extend beyond the reagent layer spatially. Thus, in one configuration, a majority of the analyte-reactive reagent is advantageously sandwiched between the optical material and the reflectance enhancing particles. A particularly effective arrangement is a textured surface on the proximal tip of an optical sensor wedge wherein both the analyte-reactive reagent and the reflectance enhancing particles are deposited within valleys or crevices in the textured surface.

Reflectance enhancing particles are normally to be applied to the surface of an optical material from an aqueous dispersion. The particles may be co-deposited on the surface along with the analyte-reactive reagent in a single step. Alternatively, the particles may be deposited in a separate step, preferably after first depositing a coating of the reagent. Drying of the coating or coatings at some point in the process is accomplished so as to present a dry sensor for handling and storage. In the case of the optical sensor device, the surface site of application would be on the wedge tips lining the inner periphery of the device annulus, followed by drying of the deposit in place on the tips.

Various chemistries may be employed as analyte-reactive reagents, and a variety of analytes in blood or other biological fluids may be assayed by use of the sensors made accordingly with the invention disclosed herein. For blood glucose, which is a commonly assayed analyte, a suitable reagent is described in the aforementioned Nomura U.S. Pat. No. 8,008,068 and is useful as well in the annular optical sensor devices described herein. More recently, blood glucose testing has evolved into testing of hemoglobin A1c (i.e. hemoglobin glycated at the N-terminal amino acid of the beta chain) as a more accurate determining factor for diagnosis of diabetes and maintenance of blood glucose control. The annular optical sensor devices of the present disclosure would be effective in such determinations when containing a suitable Hb1ac-reactive reagent on the sensor tips.

The description of the invention including its applications and advantages as set forth herein is illustrative and is not intended to limit the scope of the invention, which is set forth in the claims. Variations and modifications of the embodiments disclosed herein are possible, and practical alternatives to and equivalents of the various elements of the embodiments would be understood to those of ordinary skill in the art upon study of this patent document. Moreover, any specific values given herein are illustrative, and may be varied as desired. These and other variations and modifications of the embodiments disclosed herein, including of the alternatives and equivalents of the various elements of the embodiments, may be made without departing from the scope and spirit of the invention, including the invention as set forth in the following claims.

1. An optical sensor device for determination of an analyte in a biological fluid, comprising:
a plurality of flat optical waveguides axially arranged to form an annulus having an inner periphery and an outer periphery, each flat optical waveguide being generally in the shape of a wedge having two parallel major surfaces and an edge contiguous to the major surfaces, a portion of the edge being a tip oriented toward a central focal point of the annulus and forming a portion of the inner periphery, a portion of the edge oriented outward and forming a portion of the outer periphery of the annulus, further portions of the edge being two lateral sides of the wedge radially oriented outward from the central focal point of the annulus and partially separating the wedges one from another;
the major surfaces being coated with cladding;
each of the lateral sides being at least partially coated with cladding;
the tips having a textured surface zone; and
an analyte-reactive reagent being disposed on the textured surface zone.

2. The optical sensor device of claim 1 wherein each optical waveguide comprises an optically transparent polymer body.

3. The optical sensor device of claim 2 wherein the major surfaces, the tip, and the sides are surfaces of the optically transparent polymer body, and the cladding comprises a fluorocarbon composition.

4. The optical sensor device of claim 1 wherein the tips are beveled so as to have a slanted surface contiguous to the inner periphery.

5. The optical sensor device of claim 4 wherein the slanted surfaces are textured and an analyte-reactive reagent is disposed thereon.

6. The optical sensor device of claim 4 wherein a partial separation of the wedges is effected by cuts radiating outward from the inner periphery of the annulus.

7. The optical sensor device of claim 5 wherein the optical waveguides comprise an optically transparent polymer body.

8. The optical sensor device of claim 7 wherein the major surfaces, the tip, and the sides are surfaces of the optically transparent polymer body, and the cladding comprises a fluorocarbon composition.

9. The optical sensor device of claim 1 further comprising a plurality of particulate bodies dispersed through the analyte-reactive reagent, the particulate bodies being adapted to contribute to reflectance of light from the optical waveguide back into an optical waveguide through at least a portion of the analyte-reactive reagent, when the analyte-reactive reagent is in reaction with the analyte on the tip of at least one of the waveguides.

10. The optical sensor device of claim 1 wherein registration means is provided for mechanical engagement and movement of the device in a mechanical apparatus intended for determination of an analyte in a biological fluid.

11. An optical sensor device comprising a plurality planar wedges connected one to another in an axial arrangement to form an annulus having an inner periphery and an outer periphery, each planar wedge being partially separated from neighboring wedges by cuts made through a portion of the annulus width to form lateral sides, each wedge narrowing to a tip oriented toward a central focal point of the annulus and forming a portion of the inner periphery, a portion of the edge opposite to the tip being oriented outward and forming a portion of the outer periphery of the annulus, wherein an analyte-reactive reagent is disposed on the tips, wherein also the major surfaces are coated with cladding and each of the lateral sides is at least partially coated with cladding, wherein the tips are movable independently of one another by deflection as by means of an exterior force, an analyte-reactive reagent being resident on the tips.

12. The optical sensor element of claim 13 wherein the major surfaces, the tip, and the sides are surfaces of the optically transparent polymer body.

13. The optical sensor element of claim 14 wherein the tips forming the inner periphery are beveled so that at least a portion of the surface of each tip is at a slant to the plane of the planar wedges.

14. The optical sensor of claim 15 wherein the beveled portion of each tip is at an angle in the range of 20 to 40 degrees relative to the plane of the planar wedges.

15. The optical sensor of claim 16 wherein an analyte-reactive reagent is resident on the beveled portion of at least one of the tips.

16. The optical sensor of claim 17 further comprising a plurality of particulate bodies dispersed through the analyte-reactive reagent, the particulate bodies being adapted to contribute to reflectance of light from the planar wedge back into an planar wedge through at least a portion of the analyte-reactive reagent, when the analyte-reactive reagent is in reaction with the analyte on at least one tip of sensor.

17. The optical sensor of claim 18 wherein the beveled portion of at least one tip has been textured and an analyte-reactive reagent is resident on the textured beveled portion of the tip of the sensor.

18. The optical device of claim 13 wherein at least one registration site is provided for mechanical engagement and movement of the device in a mechanical apparatus intended for determination of an analyte in a biological fluid.

19. A method of analyzing a fluid for an analyte comprising:
mounting an annular optical sensor device having a plurality of sensor tips into a fluid testing apparatus also containing a lancet;
actuating a lancet in the apparatus so as to penetrate a surface of a living subject in order to express a fluid droplet;
positioning a tip of the optical sensor device so as to hover over the fluid droplet;
bringing the tip into contact with the fluid droplet by means of pressure from a movable shaft;
beaming a light through the optical sensor device to the tip in contact with the fluid droplet; and
measuring changes in light reflectance returning from the tip through the optical sensor device.

20. The method of claim 25 further comprising bringing a beveled tip into contact with the fluid drop.
